(19) United States
(12) Patent Application Publication
(10) Pub. No.: US 2007/0104726 A1
(43) Pub. Date: May 10, 2007

(54) Multimeric complexes of antigens and adjuvants

(75) Inventors: Pierre Andreoletti, Dijon (FR); Laurence Dumon, Saint-Beauzire (FR); Fergal Hill, Saint-Beauzire (FR); Michel Julien, Gourdon (FR); Jean Baptiste Marchand, Saint-Beauzire (FR); Emmanuel Risse, London (GB)

Correspondence Address: NIXON & VANDERHYE, PC, 901 NORTH GLEBE ROAD, 11TH FLOOR, ARLINGTON, VA, 22203, US

(73) Assignee: AVIDIS SA, Saint-Beauzire (FR)

(21) Appl. No.: 10/523,593

(22) PCT Filed: Aug. 12, 2003
(86) PCT No.: PCT/EP03/08926

§ 371 Date: Nov. 2, 2006

(30) Foreign Application Priority Data
Aug. 14, 2002 (EP) 02292042.5

(51) Int. Cl. A61K 39/29; C07H 21/04; C12N 15/86; C07K 14/02

(52) U.S. Cl. 424/189.1; 435/69.3; 435/456; 435/325; 530/350; 536/23.72

(57) ABSTRACT

The present invention provides a product comprising: a first component which is a scaffold; a second component which is an adjuvant, preferably a polypeptide which is a ligand for CD21 or a cell surface molecule on B cells or T cells or follicular dendritic or other antigen presenting cells; and a third component which is an antigen.

### INTRODUCTION

This invention relates to macromolecular assemblies, such as fusion proteins, comprising an adjuvant and an antigen, which assemblies provoke an enhanced immune response to the antigen in comparison to the antigen alone.

### BACKGROUND OF THE INVENTION

Adjuvants enhance the immune response to antigens and are therefore useful in vaccines. However, there are only a limited number of adjuvants approved for use in humans, and as stronger adjuvants are known from research on animals, a clear need exists for stronger immunological adjuvants which are safe to use in man. For a recent review, see “Advances in vaccine adjuvants” (Nature Biotechnology, 1999, Volume 17, pages 1075-1081). A critical feature of any adjuvant for widespread use in man is that it should be very safe, particularly if it is to be used in routine prophylaxis in very large numbers of healthy people.

The complement system consists of a set of serum proteins that are important in the response of the immune system to foreign antigens. The complement system becomes activated when its primary components are cleaved and the products, alone or with other proteins, activate additional complement proteins resulting in a proteolytic cascade. Activation of the complement system leads to a variety of responses including increased vascular permeability, chemotaxis of phagocytic cells, activation of inflammatory cells, opsonisation of foreign particles, direct killing of cells and tissue damage.

Activation of the complement system may be triggered by antigen-antibody complexes (the classical pathway) or a normal slow activation may be amplified in the presence of cell walls of invading organisms such as bacteria and viruses (the alternative pathway). The complement system interacts with the cellular immune system through a specific pathway involving C3, a protein central to both classical and alternative pathways. The proteolytic activation of C3 gives rise to a large fragment (C3b) and exposes a chemically reactive internal thiolester linkage which can react covalently with external nucleophiles such as the cell surface proteins of invading organisms or foreign cells. As a result, the potential antigen is “tagged” with C3b and remains attached to that protein as it undergoes further proteolysis to iC3b and C3d,g. The latter fragments are, respectively, ligands for the complement receptors CR3 and CR2; (CR2 is also referred to as CD21). Thus the labelling of antigen by C3b can result in a targeting mechanism for cells of the immune system bearing these receptors.

That such targeting is important for augmentation of the immune response is first shown by experiments in which mice were depleted of circulating C3 and then challenged with an antigen (sheep erythrocytes). Removal of C3 reduced the antibody response to this antigen (M. B. Pepys, J. Exp. Med., 140, 126-145, 1974). The role of C3 was confirmed by studies in animals genetically deficient in either C3 or the upstream components of the complement cascade which generate C3b, i.e. C2 and C4 (J. M. Ahearn and D. T. Fearon, Adv. Immunol., 46, 183-219, 1989). More recently, it has been shown that linear conjugation of a model antigen with more than two copies of the murine C3d fragment sequence resulted in a very large (1000-10000-fold) increase in antibody response in mice compared with unmodified antigen controls (P. W. Dempsey et al, Science, 271, 348-350, -1996; WO96/17625, PCT/GB95/02851). The increase could be produced without the use of conventional adjuvants such as Freund's complete adjuvant, which is too toxic to be used in humans. The mechanism of this remarkable effect was demonstrated to be high-affinity binding of the multivalent C3d construct to CR2 on B-cells, followed by co-ligation of CR2 with another B-cell membrane protein, CD19 and with membrane-bound immunoglobulin to generate a signal to the B-cell nucleus.

However, it has proved difficult to produce large amounts of homogenous recombinant proteins containing three copies of C3d. The principal problems have been:
i) the genetic instability of the constructs containing (three) repeated sequences and
ii) the folding (or solubilisation and refolding) of the recombinant protein from inclusion bodies formed in Escherichia coli.

One approach taken to minimise the genetic instability of constructs containing repeated copies of the C3d gene is described in WO99/35260 and WO01/77324. The technology described in these applications is to use non-identical sequences of DNA encoding repeats of C3d.

WO00/69907 and WO00/69886, the contents of which are incorporated herein by reference, describe polypeptide monomers capable of assembling into a multimeric form. The monomers are derived from chaperone proteins, particularly GroES or Cpn10 family members.

A multimerisation system using the complement 4 binding protein (C4bp) is described in WO 91/11461. Human C4b-binding protein (C4BP) is a plasma glycoprotein of high molecular mass (570 kDa) which has a spider like structure made of seven identical alpha-chains and a single beta-chain. The C4bp alpha chain has a C-terminal core region responsible for assembly of the molecule into a multimer. According to the standard model, the cysteine at position +498 of one C4bp monomer forms a disulphide bond with the cysteine at position +510 of another monomer. A minor form comprising only seven alpha-chains has also been found in human plasma. The natural function of this plasma glycoprotein is to inhibit the classical pathway of complement activation.

WO 91/11461 proposes that the ability of the C4bp protein to multimerise can be used to make fusion proteins comprising all or part of C4bp and a biological protein of interest. The fusion protein will form multimers which provides a platform for the protein of interest, in which said protein has an enhanced serum half-life and increased affinity or avidity for its targets. Fusion proteins of C4bp were targeted as the focus of novel delivery and carrier systems for therapeutic products in WO 91/11461.

Most of the alpha-chain of C4bp is composed of eight tandemly arranged domains of approximately 60 amino acids in length known as complement control protein (CCP) repeats. Inclusion of one or more of these domains was preferred in the fusion proteins described in WO 91/11461, but it has since been demonstrated that all CCPs can be deleted (leaving only the C-terminal 57 amino acids) without preventing multimerisation (Libyh M. T. et al., (1997) Blood, 90, 3978-3983). This C-terminal region of C4bp is referred to as the C4bp core.

Libyh et al., (1997), describe a protein multimerisation system which is based on the C-terminal part of the alpha chain of C4bp. The C-terminal part of the C4bp lacks lacks the ability to inhibit the classical pathway of complement activation, but is responsible for polymerisation of C4bp in the cytoplasm of CHO cells producing C4bp. Libyh et al. were able to induce spontaneous multimerisation of associated antibody fragments to create homomultimers of scFv fragments using the C4bp fragment. The C-terminal portion of C4bp used was placed C-terminal to the scFv sequence, optionally spaced by a MYC tag.

The use of C4bp is also described in Oudin et al. (2000, Journal of Immunology, Vol. 164:1505) and Christiansen et al. (2000, Journal of Virology, Vol. 74:4672). Self-assembling multimeric soluble CD4-C4bp fusion protein have also been demonstrated in Shinya et al (1999, Biomed & Pharmacother, Vol. 53: 471) where the fusion proteins were expressed in the human 293 cell line.

### SUMMARY OF THE INVENTION

The present invention provides a product comprising:
a first component which is a scaffold;
a second component which is an adjuvant, preferably a polypeptide which is a ligand for CD21 or a cell surface molecule on B cells or T cells or follicular dendritic or other antigen presenting cells; and
a third component which is-an antigen.

The first component provides for assembly of multiple copies of the second component in a multi-component product such that the multiple copies of the second component are associated with one or more copies of the antigen.

In a preferred aspect, the invention provides:
a first component which is a polypeptide scaffold;
a second component which is a polypeptide which is a ligand for CD21 or a cell surface molecule on B cells or T cells or on follicular dendritic or other antigen presenting cells; and
a third component which is an antigen.

The first and second components may be in the form of a fusion protein. When the third component is also a polypeptide, the three components are present as a fusion protein. Alternatively the third component is covalently linked to a fusion of the first two components.

In some cases, where the first component is itself an antigen, the first and third components may be the same molecule.

For the avoidance of doubt, the designation of “first”, “second” and “third” components does not imply or indicate a specific linear order in the product of the three components. The three components may be joined in any order.

Thus where all three components are polypeptides and the product is made as a fusion protein, the N— to C-terminal order of the three components may be in any permutation. Further, as indicated below, in some cases the first component may include loop regions which can be replaced by one or other of the second and third components.

The product of the present invention provides for the immunostimulatory second component to be formed into a multi-component product, and to be expressed using recombinant DNA technology without the need to use DNA sequences having tandem repeat sequences.

The invention further provides nucleic acid encoding a fusion protein of said first and second components and, where said third component is a polypeptide, nucleic acid encoding all three components. The invention also provides vectors comprising said nucleic acids and host cells carrying said vectors.

In another embodiment, the invention provides a method of making a product comprising:
a first component which is a polypeptide scaffold;

1a second component which is a polypeptide which is a ligand for CD21 or a cell surface molecule on B cells or T cells or follicular dendritic or other antigen presenting cells; and
a third component which is a polypeptide antigen, the method comprising expressing nucleic acid encoding the three components in the form of a fusion protein, and recovering the product.

In another embodiment, the invention provides a method of making a product comprising:
a first component which is a polypeptide scaffold;
a second component which is a polypeptide which is a ligand for CD21 or a cell surface molecule on B cells or T cells or follicular dendritic or other antigen presenting cells; and
a third component which is a non-polypeptide antigen, the method comprising expressing nucleic acid encoding the first and second components in the form of a fusion protein, joining said fusion protein to the third component, and recovering the product.

The methods of making the product may be performed in eukaryotic or prokaryotic cells.

The invention also provides a method of inducing an immune response to an antigen which method comprises administering to a subject an effective amount of a product according to the invention.

The invention also provides the use of a product of the invention for a method of treatment of the human or animal body, particularly a method of inducing an immune response.

The invention further provides a pharmaceutical composition comprising a product of the invention in association with a pharmaceutically acceptable carrier or diluent.

### DESCRIPTION OF THE DRAWINGS

FIG. 1 shows an alignment of C4bp core proteins.

FIG. 2 shows the binding of the epitope-C3d-C4bp fusion protein and of C3d7(1)to CR2 (also known as CD21) in comparison to monomeric C3d and a linear trimeric version of C3d, called C3d3.

FIG. 3 is a cartoon representing the format of the CR2 binding assay.

FIG. 4 shows the binding of C3d7(1) to CR2 CD21) in comparison to monomeric C3d and a linear trimeric version of C3d, called C3d3.

FIG. 5 shows the binding of C3d7(1), (2) and (3) to CR2 (CD21) in comparison to monomeric C3d and a linear trimeric version of C3d, called C3d3.

FIG. 6 shows a flow cytometry analysis of C3d7(1), C3d7(2) and C3d7(3) binding to Raji and Jurkat cells.

### DETAILED DESCRIPTION OF THE INVENTION

Scaffold

This refers to any macromolecular assembly which is capable of being a scaffold to which the second and third components may be attached. It may be a protein or other polymeric molecule (composed for example of sugars) or a prokaryotic or eukaryotic cell wall or a virus. The cell walls, or viruses or proteins may be incomplete, that is lacking components normally present in the organism in which it is found; the important feature for this invention is that the scaffold is capable of uniting into a single assembly more than one adjuvant molecule and more that one antigen molecule.

As is described in more detail herein, there are two main classes of scaffold contemplated. The first is a complex macromolecular product, including a virus or cell, onto which multiple copies of the second and, where applicable, third component are attached, either separately or as a fusion of the second and third components. Alternatively, the scaffold is present in a 1:1 ratio with the second component. When the product is in the form of a fusion protein then the third component is also present in a 1:1:1 ratio with the second and first components.

Cell wall or viral scaffolds are known in the art for other purposes. Surface display of proteins, whether on prokaryotic (Samuelson et al., 2002, J. Biotechnol 96,129-154; Lang H., 2001, Nat. Biotechnol., 19, 75-78) or eukaryotic cell walls (Shusta E. V. et al., 1999, J. Mol. Biol. 292, 949-956) or viruses, such as bacteriophages (Sidhu S. S., 2001, Biomol. Eng., 18, 57-63) have been described. The distinctive feature of this aspect of the invention is that the objects displayed on the cell wall include more than one copy of an adjuvant molecule simultaneously present with an antigen. The antigen may be fused directly to the adjuvant (such as C3d), but need not be.

Thus in one embodiment, the surface of a cell, such as a bacterium, can serve as the scaffold. When the adjuvant is fused genetically to a second component normally expressed on the surface of the bacterium, multiple copies of the adjuvant are displayed on the surface of the bacterium. This has the effect of eliciting an improved immune response against the bacterium, when the bacterium infects a host. The antigen may be the cell wall of the bacterium, or an antigen separately but simultaneously expressed on the surface of the bacterium. The infection may be deliberate, by the administration of the modified bacterium to the host. The bacterium may be administered either after being killed, or in a live but attenuated form.

Similarly, eukaryotic cells may be used as the scaffold. In such a case, the cell surface displaying more than one copy of the adjuvant can be used to elicit an immune response to other (normal or abnormal) cell surface components.

In contrast to the fusion proteins described in WO96/17625, (PCT/GB95/02851), there need not be a covalent linkage either at all between the antigen and adjuvant, or the covalent linkage may only be indirect, being mediated by the scaffold. Furthermore, WO96/17625 teaches that the fusion of a single copy of the C3d protein to an antigen decreases the immune response to that antigen. In this invention, in direct contrast, either the display of multiple single (monomeric) copies of the adjuvant, or the fusion of a single copy of the antigen to a single copy of the adjuvant, which are then fused to a scaffold, results in an increased immune response to the antigen.

The antigen may be the cell wall itself or a second protein or glycoprotein. In the case of organisms where the protective antigen is the capsule, as in the case of pneumococci, the display of more than one copy of the adjuvant will improve the immune response to the capsular antigens.

In the case of viruses, the antigen may be the virus itself, which thus acts simultaneously as antigen and scaffold. An example is provided of the hepatitis B virus surface antigen. Methods for preparing a recombinant HBsAg vaccine are described in U.S. Pat. No. 4,769,238. Although this recombinant HBsAg is a very successful vaccine, there remain a substantial number of vaccine recipients who are “poor responders”. The addition of a new adjuvant to this existing vaccine will enable the vaccination of such poor responders, and the post-infection vaccination of chronic carriers of this virus. One method envisaged of adding the adjuvant to this vaccine is the genetic fusion of the coding sequence of the an adjuvant protein, such as and preferably the human C3d protein, to the C-terminus of the gene encoding the 226 amino acid residue protein that is the S protein of the hepatitis B virus. The coding sequence for the adjuvant can be added, optimally with codons preferred for high-level expression in yeast, in-frame to the S protein coding sequence present in the plasmids described in the U.S. Pat. No. 4,769,238 referred to above. The sequence of the S protein may be modified to include variant sequences, known as “escape mutants” (Cooreman M. P. et al., 2001, J. Biomed. Sci. 8, 237-247) or antigens not normally found in the hepatitis B vaccine (Fomsgaard A. et al. 1998, Scand. J. Immunol., 47, 289-295). As described in that article, the modified vaccine containing the C3d adjuvant can be administered as DNA in order to obtain an immune response.

Thus in another embodiment, the polypeptide scaffold may be itself an antigen. Thus the surface antigen of hepatitis B virus, which assembles into oligomeric structures, can simultaneously be the first and third component of the invention. As first remarked on in 1956 (F H C Crick, J D Watson, Nature, 177, 473) the finite nucleic acid content of viruses severely restricts the number of amino acids that viruses can encode. As a consequence, the protein coat can not be constructed from a very large number of different protein molecules. Instead it must be constructed from a number of identical small sub-units arranged in a regular manner. Thus most viruses will be capable of simultaneously being both the first and third component of the invention.

A polypeptide scaffold is a protein, or part thereof, whose function is to determine the structure of the protein itself, or of a group of associated proteins or other molecules. Polypeptide scaffolds therefore have a defined three-dimensional structure when assembled, and have the capacity to support molecules or polypeptides—in or on the said structure. Advantageously, a scaffold has the ability to assume a variety of viable geometries, in relation to the three-dimensional structure of the scaffold and/or the insertion site of the polypeptides.

In another embodiment, the scaffold may serve as the adjuvant, i.e. the first and second components will be the same. The scaffold which is an adjuvant may be a C4bp core protein or a fragment of the C4bp alpha chain, described in further detail herein.

In one embodiment, the scaffold is a cochaperonin Cpn10/Hsp10 scaffold. Cpn10 is a widespread component of the Cpn60/Cpn10 chaperonin system. Examples of Cpn10 include human mitochondrial Cpn10, bacterial GroES and bacteriophage T4 Gp31. Further members of the Cpn10 family will be known to those skilled in the art.

The invention moreover comprises the use of derivatives of naturally-occurring scaffolds. Derivatives of scaffolds (including scaffolds of the Cpn10 and 60 families) comprise mutants thereof, which may contain amino acid deletions, additions or substitutions (especially replacement of Cys residues in Gp31), hybrids formed by fusion of different members of the Cpn10 or Cpn60 families and/or circular permutated protein scaffolds, subject to the maintenance of the “oligomerisation” property described herein.

Polypeptide scaffolds assemble to form a multimeric product. In the context of the present invention, the multimeric product may have any shape and may comprise any number of individual scaffold units.

Preferably, the mutimeric product comprises between 2 and 20 scaffold units, advantageously between 5 and 15 units, and ideally about 10 units. The scaffold of Cpn10 family members comprises seven protein units, in the shape of a seven-membered ring or annulus. Advantageously, therefore, the multimeric product is a seven-membered ring.

It is known that Cpn10 subunits possess a “mobile loop” within their structure. The mobile loop is positioned between amino acids 15 and 34, preferably between amino acids 16 to 33, of the sequence of Escherichia coli GroES, and equivalent positions on other members of the Cpn10 family. The mobile loop of T4 Gp31 is located between residues 22 to 45, advantageously 23 to 44. The polypeptide sequence of the second or third component may be inserted by replacing all or part of the mobile loop of a Cpn10 family polypeptide.

Where the polypeptide scaffold is a Cpn10 family polypeptide, the second or third component polypeptide may moreover be incorporated at the N or C terminus thereof, (which terminus may be the natural or a modified N or C terminus) or in positions which are equivalent to the roof beta hairpin of Cpn10 family peptides. This position is located between positions 54 and 67, advantageously 55 to 66, and preferably 59 to 61 of bacteriophage T4 Gp31, or between positions 43 to 63, preferably 44 to 62, advantageously 56 to 57 of E. coli GroES.

In another embodiment, the polypeptide scaffold may be a C4bp protein or part thereof retaining the C4bp core protein region.

Human C4 binding protein (hC4bp) is a molecule possessing many attractive characteristics as a delivery vehicle for bioactive molecules. Human C4bp is involved in the human complement system—a group of immune system proteins whose functions include lysing invading cells, activating phagocytic cells and facilitating the clearance of foreign substances from the system. It regulates the activity of proteins in this system, particularly C4 protein. Structurally, hC4bp is a flexible, disulfide-bonded molecule expected to have long serum half-life and the ability to target bioactive molecules to the lymph nodes. The serum form of hC4bp has a molecular weight of about 590 kD. On reducing SDS gels, hC4bp produces a strong band at about 70 kD, indicating a disulfide-bonded multimeric protein.

A cDNA encoding the C4bp monomer has been cloned and characterized [L. P. Chung et al., (1985) “Molecular Cloning and Characterization of the cDNA Coding for C4b-Binding Protein of the Classical Pathway of the Human Complement System”, Biochem. J., 230, 133-141 ]. Chung et al. refers to hC4bp as a polypeptide of 549 amino acids. The polypeptide predicted from the DNA sequence has a molecular weight of about 61.5 kD, rather than 70 kD as actually measured on reducing SDS gels. The difference in molecular weight apparently is due to glycosylation of the serum form of the polypeptide. The first 491 amino acids from the N-terminus of the Chung et al. sequence are divisible into eight domains called short consensus repeat regions (SCRs) of about sixty amino acids each. These regions are designated, from N-terminus to C-terminus, SCR8 to SCR1. The SCR domains are defined as follows: SCR8-+1 to +61; SCR7-+62 to +123; SCR6-+124 to +187; SCR5-+188 to +247; SCR4-+248 to +313; SCR3-+314 to +374; SCR2-+375 to +432; SCR1-+433 to +491. These domains, which share significant sequence homology, each contain four similarly situated cysteine residues. These cysteine residues form intra-domain disulfide bonds in a regular pattern [J. Janatova et al., (1989) “Disulfide Bonds re Localized Within the Short Consensus Repeat Units of Complement Regulatory Proteins: C4b-Binding Protein”, Biochemistry, 28, 4754-4761 ]. Within each SCR domain, the first cysteine residue bonds with the third and the second cysteine residue bonds with the fourth, forming a double-loop amino acid sequence. Thus, the SCRs are connected like beads on a string. This pattern of intra-domain disulfide bonding is responsible for the conformational flexibility of the C4bp monomer. In addition to the eight SCR domains, hC4bp also has a 57 amino acid sequence at the C-terminus, the C4bp core, which bears no homology to the other regions of the protein. This region is responsible for assembly of the molecule into a multimer.

Thus the polypeptide scaffold may be a C4bp core and optionally one or more SCRs fused to the core.

In a particularly preferred embodiment, the polypeptide scaffold is the core protein of C4bp alpha chain.

A polypeptide scaffold may additionally comprise N— or C-terminal extensions such as flexible linkers such as (Glyₘ-Ser)ₙ (where m and n-are from 1 to 4). These are used in the art to attach protein domains (particularly antibody V domains) to each other. Thus the first component may be linked to the second and/or third component by such a linker.

It is preferred that the first component is at the C-terminal of the product, when the core protein of C4bp alpha chain is the scaffold.

Core Protein of C4bp Alpha Chain.

This is referred to herein as the “C4bp core protein” or “core protein”, or “C4bp scaffold”. The terms are used interchangeably. This protein may be a mammalian C4bp core protein or a fragment thereof capable of forming multimers, or a synthetic variant thereof capable of forming multimers.

The sequences of a number of mammalian C4bp proteins are available in the art. These include human C4bp core protein (SEQ ID NO:l). There are a number of homologues of human C4bp core protein available in the art. There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

For example, a search of GenBank indicates mammalian C4bp core homologue proteins in species including rabbit, rat, mouse and bovine origin (SEQ ID NO:2-5 respectively). Paralogues have been identified in pig (ApoR), guinea pig (AM67) and mouse (ZP3); shown as SEQ ID NO:6-8 respectively.

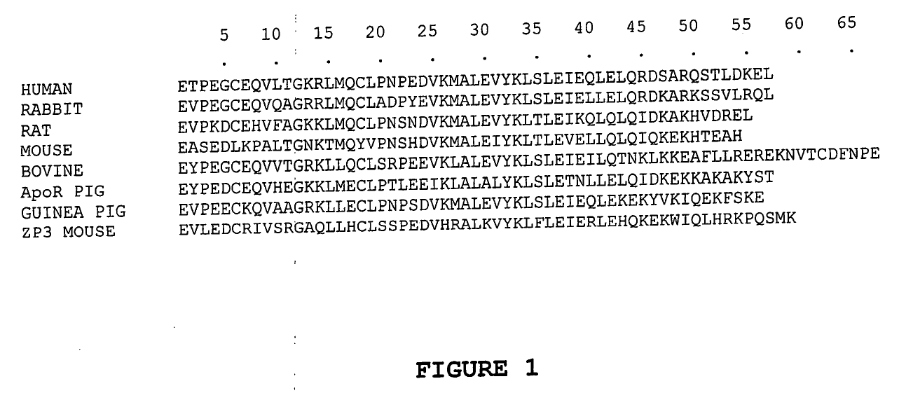

An alignment of SEQ ID NOs:1-8 is shown as FIG. 1. It can be seen that all eight sequences have a high degree of similarity, though with a greater degree of variation at the C-terminal end. Further C4bp core proteins may be identified by searching databases of DNA or protein sequences, using commonly available search programs such as BLAST.

Where a C4bp protein from a desired mammalian source is not available in a database, it may be obtained using routine cloning methodology well established in the art. In essence, such techniques comprise using nucleic acid encoding one of the available C4bp core proteins as a probe to recover and to determine the sequence of the C4bp core proteins from other species of interest. A wide variety of techniques are available for this, for example PCR amplification and cloning of the gene using a suitable source of mRNA (e.g. from an embryo or an actively dividing differentiated or tumour cell), or by methods comprising obtaining a cDNA library from the mammal, e.g. a cDNA library from one of the above-mentioned sources, probing said library with a known C4bp nucleic acid under conditions of medium to high stringency (for example 0.03M sodium chloride and 0.03M sodium citrate at from about 50° C. to about 60° C.), and recovering a cDNA encoding all or part of the C4bp protein of that mammal. Where a partial cDNA is obtained, the full length coding sequence may be determined by primer extension techniques.

A fragment of a C4bp core protein capable of forming multimers may comprise at least 47 amino acids, preferably at least 50 amino acids. The ability of the fragment to form multimers may be tested by expressing the fragment in a prokaryotic host cell according to the invention, and recovering the C4bp fragment under conditions which result in multimerisation of the full 57 amino acid C4bp core, and determining whether the fragment also forms multimers. Desirably a fragment of C4bp core comprises at least residues 6-52 of SEQ ID NO:1 or the corresponding residues of its homologues.

The human C4bp core protein of SEQ ID NO:1 corresponds to amino acids +493 to +549 of full length C4bp protein sequence. A fragment of this known in the art to form multimers corresponds to amino acids +498 to +549 of C4bp core protein.

Variants of C4bp core and fragments capable of forming multimers, which variants likewise retain the ability to form multimers (which may be determined as described above for fragments) may also be used. The variant will preferably have at least 70%, more preferably at least 80%, even more preferably at least 90%, for example at least 95% or most preferably at least 98% sequence identity to a wild type mammalian C4bp core or a multimer-forming fragment thereof. In one aspect, the C4bp core will be a core which includes the two cysteine residues which appear at positions 6 and 18 of SEQ ID Nos:1-3 and 5-8. Desirably, the variant will retain the relative spacing between these two residues.

The above-specified degree of identity will be to any one of SEQ ID NOs:1-8 or a multimer-forming fragment thereof.

Most preferably the specified degree of identity will be to SEQ ID NO:1 or a multimer-forming fragment thereof.

The degree of sequence identity may be determined by the algorithm GAP, part of the “Wisconsin package” of algorithms widely used in the art and available from Accelrys (formerly Genetics Computer Group, Madison, Wis.). GAP uses the Needleman and Wunsch algorithm to align two complete sequences in a way that maximises the number of matches and minimises the number of gaps. GAP is useful for alignment of short closely related sequences of similar length, and thus is suitable for determining if a sequence meets the identity levels mentioned above. GAP may be used with default parameters.

Synthetic variants of a mammalian C4bp core protein include those with one or more amino acid substitutions, deletions or insertions or additions to the C- or N-termini. Substitutions are particularly envisaged. Substitutions include conservative substitutions. Examples of conservative substitutions include those set out in the following table, where amino acids on the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |
| OTHER | | N Q D E |

Examples of fragments and variants of the C4bp core protein which may be made and tested for their ability to form multimers thus include SEQ ID NOs: 9 to 16, shown in Table 1 below:

| | | |
|---|---|---|
| A | B | C |

| | | |
|---|---|---|
| 9 | -----CEQVLTGKRLMQCLPNPEDVKMALEVYKLSLEIEQLELQRDSARQSTLDKEL | 100 |
| 10 | ETPEGCEQVLTGKRLMQCLPNPEDVKMALEVYKLSLEIKQLELQRDSARQSTLDKEL | 98 |
| 11 | -----CEQVLTGKRLMQCLPNPEDVKMALEVYKLSLEIKQLELQRDSARQSTLDKEL | 98 |
| 12 | ETPEGCEQVLTGKRLMQCLPNPEDVKMALEIYKLSLEIEQLELQRDSARQSTLDKEL | 98 |
| 13 | ETPEGCEQVLTGKRLMQCLPNPEDVKMALEIYKLSLEIKQLELQRDSARQSTLDKEL | 96.5 |
| 14 | ---EGCEQALTGKRLMQCLPNPEDVKMALEIYKLSLEIKQLELQRDSARQSTL---- | 94 |
| 15 | ETPEGSEQVLTGKRLMQSLPNPEDVKMALEVYKLSLEIKQLELQRDSARQSTLDKEL | 94 |
| 16 | ---EGSEQALTGKRLMQSLPNPEDVKMALEIYKLSLEIEQLELQRDSARQSTLDK-- | 92.3 |

A = SEQ ID NO:; B = sequence, C = % identity, calculated by reference to a fragment of SEQ ID NO: 1 of the same length.

Where deletions of the sequence are made, apart from N- or C-terminal truncations, these will preferably be limited to no more than one, two or three deletions which may be contiguous or non-contiguous.

Where insertions are made, or N- or C-terminal extensions to the core protein sequence, these will also be desirably limited in number so that the size of the core protein does not exceed the length of the wild type sequence by more than 20, preferably by more than 15, more preferably no more than 10, amino acids. Thus in the case of SEQ ID NO:1, the core protein, when modified by insertion or elongation, will desirably be no more than 77 amino acids in length.

Second Component.

The product of the invention will comprise the scaffold as described above linked to the second component either directly or indirectly and the third component.

The second component may be any ligand for CD21 or CD19, as described in U.S. Pat. No. 6,238,670, and WO99/35260, the contents of which are hereby incorporated by reference. The second component may also be a ligand for a cell surface molecule on B cells or T cells or follicular dendritic or other antigen presenting cells.

Preferably, the second component is C3d, particularly human C3d.

The nucleotide sequence and predicted amino acid sequence of mouse C3d are disclosed in Domdey et al. (1982) Proc. Natl. Acad. Sci. USA 79: 7619-7623 and Fey et al. (1983) Ann. N.Y. Acad. Sci. 421: 307-312). The nucleotide sequence and predicted amino acid sequence for human C3d are disclosed in de Bruijn and Fey (1985) Proc. Natl. Acad. Sci. USA 82:708-712. Nucleic acid encoding C3d from other species may be isolated using the human or mouse sequence information to prepare one or more probes for use in standard hybridisation methods. When C3d is to be employed in the invention and administered to a subject, the C3d may be matched to the species to be immunised (e.g. mouse C3d to be used in mouse, human C3d in human and so on). Furthermore, the codons chosen may also be optimised for the species to be immunised, for example using codons that are efficiently translated in mammalian hosts.

Where the second component is linked by a peptide linker to the first and/or third component, the linker may be a flexible linker as described above.

In a preferred embodiment, the second component is N-terminal to the first component, and C-terminal to the antigen (where the antigen is a polypeptide) when the scaffold is the C4bp core protein. Where the antigen is not a polypeptide, the antigen may be covalently linked to either of the first or second components.

Antigen.

Antigens may be any product of prophylactic value; they might be useful for vaccination. The invention allows rapid progress from nucleotide sequences to the production of recombinant antigens attached to an adjuvant in a polyvalent form.

Bacterial immunogens, parasitic immunogens and viral immunogens are useful as polypeptide moieties to create multimeric or hetero-multimeric C4bp fusion proteins useful as vaccines.

Bacterial sources of these immunogens include those responsible for bacterial pneumonia, pneumocystis pneumonia, meningitis, cholera, tetanus, tuberculosis and leprosy.

Parasitic sources include malarial parasites, such as Plasmodium.

Viral sources include poxviruses, e.g., cowpox virus and orf virus; herpes viruses, e.g., herpes simplex virus type 1 and 2, B-virus, varicellazoster virus, cytomegalovirus, and Epstein-Barr virus; adenoviruses, e.g., mastadenovirus; papovaviruses, e.g., papillomaviruses such as HPV16, and polyomaviruses such as BK and JC virus; parvoviruses, e.g., adeno-associated virus; reoviruses, e.g., reoviruses 1, 2 and 3; orbiviruses, e.g., Colorado tick fever; rotaviruses, e.g., human rotaviruses; alphaviruses, e.g., Eastern encephalitis virus and Venezuelan encephalitis virus; rubiviruses, e.g., rubella; flaviviruses, e.g., yellow fever virus, Dengue fever viruses, Japanese encephalitis virus, Tick-borne encephalitis virus and hepatitis C virus; coronaviruses, e.g., human coronaviruses; paramyxoviruses, e.g., parainfluenza 1, 2, 3 and 4 and mumps; morbilliviruses, e.g., measles virus; pneumovirus, e.g., respiratory syncytial virus; vesiculoviruses, e.g., vesicular stomatitis virus; lyssaviruses, e.g., rabies virus; orthomyxoviruses, e.g., influenza A and B; bunyaviruses e.g., LaCrosse virus; phleboviruses, e.g., Rift Valley fever virus; nairoviruses, e.g., Congo hemorrhagic fever virus; hepadnaviridae, e.g., hepatitis B; arenaviruses, e.g., 1 cm virus, Lasso virus and Junin virus; retroviruses, e.g., HTLV I, HTLV II, HIV-1 and HIV-2; enteroviruses, e.g., polio virus 1,- 2 and 3, coxsackie viruses, echoviruses, human enteroviruses, hepatitis A virus, hepatitis E virus, and Norwalk-virus; rhinoviruses e.g., human rhinovirus; and filoviridae, e.g., Marburg (disease) virus and Ebola virus.

Antigens from these bacterial, viral and parasitic sources may be used in the production of multimeric proteins useful as vaccines. The multimers may comprise a mixture of monomers carrying different antigens.

Immunogens to human proteins for research or therapeutic purposes may be made. These have many applications not only in vaccination but also in research. For example, the generation of human gene sequence data by the human genome project has made the generation of antisera reactive to new polypeptides a pressing requirement. The same requirement applies to prokaryotic, such as bacterial, and other eukaryotic, including fungal, gene products.

Non-polypeptide immunogens may be, for example, carbohydrates or nucleic acids. The polysaccharide coats of Neisseria species or of Streptococcus pneumoniae species are examples of carbohydrates which may be used for the purposes of the invention.

The antigen may be any size conventional in the art for vaccines, ranging from small polypeptides to larger proteins. Due to the nature of the present invention, antigens of up to 100 kDa, and more preferably up to 50 kDa, such as up to 30 kDa in size are preferred.

Where a non-polypeptide immunogen is part of the product of the invention, the immunogen may be covalently attached to the first and second components of the product using routine synthetic methods. Generally, the immunogen may be attached to either the N— or C-terminal of a fusion protein comprising the first and second components, or to an amino acid side chain group (for example the epsilon-amino group of lysine), or a combination thereof. More than one immunogen per fusion protein may be added. To facilitate the coupling, a cysteine residue may be added to the fusion protein, for example as the C-terminus.

The present invention has many advantages in the generation of an immune response. For example, the use of multimers can permit the presentation of a number of antigens, simultaneously, to the immune system. This allows the preparation of polyvalent vaccines, capable of raising an immune response to more than one epitope, which may be present on a single organism or a number of different organisms. Thus, vaccines formed according to the invention may be used for simultaneous vaccination against more than one disease, or to target simultaneously a plurality of epitopes on a given pathogen. The epitopes may be present in a single monomer units or on different monomer units which are combined to provide a heteromultimer.

Human C4bp core fusion proteins or human Cpn10 fusion proteins in particular are useful in the context of immunisations, because the core protein and human Cpn10 are not only present normally in the serum or plasma of the recipient of the immunisation, but also because they do not themselves evoke an immune response. C4bp proteins are known in a number of mammalian species, and the appropriate homologues for mammalian species may be found by those skilled in the art using standard gene cloning techniques.

Nucleic Acids.

Products of the invention may be produced by expression of a fusion protein of at least the first and second components in a prokaryotic or eukaryotic host cell, using a nucleic acid construct encoding the protein. Where the third component is a polypeptide, the expression of all three components from a nucleic acid sequence can be used to produce a product of the invention.

Thus the invention provides a nucleic acid construct, generally DNA or RNA, which encodes a product of the invention.

The construct will generally be in the form of a replicable vector, in which sequence encoding the protein is operably linked to a promoter suitable for expression of the protein in a desired host cell.

The vectors may be provided with an origin of replication and optionally a regulator of the promoter. The vectors may contain one or more selectable marker genes. There are a wide variety of prokaryotic and eukaryotic expression vectors known as such in the art, and the present invention may utilise any vector according to the individual preferences of those of skill in the art.

A wide variety of prokaryotic host cells can be used in the method of the present invention. These hosts may include strains of Escherichia, Pseudomonas, Bacillus, Lactobacillus, Thermophilus, Salmonella, Enterobacteriacae or Streptomyces. For example, if E. coli from the genera Escherichia is used in the method of the invention, preferred strains of this bacterium to use would include BL21(DE3) and their derivatives including C41(DE3), C43(DE3) or C0214(DE3), as described and made available in WO98/02559.

Even more preferably, derivatives of these strains lacking the prophage DE3 may be used when the promoter is not the T7 promoter.

Prokaryotic vectors includes vectors bacterial plasmids, e.g., plasmids derived from E. coli including ColEI, pCR1, pBR322, pMB9 and their derivatives, wider host range plasmids, e.g., RP4; phage DNAs, e.g., the numerous derivatives of phage A, e.g., NM989, and other DNA phages, e.g., M13 and filamentous single stranded DNA phages. These and other vectors may be manipulated using standard recombinant DNA methodology to introduce a nucleic acid of the invention operably linked to a promoter.

The promoter may be an inducible promoter. Suitable promoters include the T7 promoter, the tac promoter, the trp promoter, the lambda promoters P_{L} or P_{R} and others well known to those skilled in the art.

A wide variety of eukaryotic host cells may also be used, including for example yeast, insect and mammalian cells. Mammalian cells include CHO and mouse cells, African green monkey cells, such as COS-1, and human cells.

Many eukaryotic vectors suitable for expression of proteins are known. These vectors may be designed to be chromosomally incorporated into a eukaryotic cell genome or to be maintained extrachromosomally, or to be maintained only transiently in eukaryotic cells. The nucleic acid may be operably linked to a suitable promoter, such as a strong viral promoter including a CMV promoter, and SV40 T-antigen promoter or a retroviral LTR.

To obtain a product of the invention, host cells carrying a vector of the invention may be cultured under conditions suitable for expression of the protein, and the protein recovered from the cells of the culture medium.

Compositions

Products according to the invention may be prepared in the form of a pharmaceutical composition. The product will be present with one or more pharmaceutically acceptable carriers or diluents. The composition will be prepared according to the intended use and route of administration of the product. Thus the invention provides a composition comprising a product of the invention in multimeric form together with one or more pharmaceutically acceptable carriers or diluents, and the use of such a composition in methods of immunotherapy for treatment or prophylaxis of a human or animal subject.

Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

Liquid pharmaceutically administrable compositions can, for example, be prepared by dissolving, dispersing, etc, a fusion protein of the invention optional pharmaceutical adjuvants in a carrier, such as, for example, water, saline aqueous dextrose, glycerol, ethanol, and the like, to thereby form a solution or suspension. If desired, the composition to be administered may also auxiliary substances such as pH buffering agents and the like. Actual methods of preparing such dosage forms are known, or will be apparent, to those skilled in this art; for example, see Remington's Pharmaceutical Sciences, Mack Publishing Company, Easton, Pa., 19th Edition, 1995.

The composition or formulation to be administered will, in any event, contain a quantity of the active compound(s) in an amount effective to alleviate the symptoms of the subject being treated. Dosage forms or compositions containing active ingredient in the range of 0.25 to 95% with the balance made up from non-toxic carrier may be prepared.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. A more recently devised approach for parenteral administration employs the implantation of a slow-release or sustained-release system, such that a constant level of dosage is maintained. See, e.g., U.S. Pat. No. 3,710,795.

Doses of the product will be dependent upon the nature of the antigen and may be determined according to current practice for administration of that antigen in conventional vaccine formulations.

DNA Vaccines

In another aspect, the invention provides a eukaryotic expression vector comprising a nucleic acid sequence encoding a recombinant fusion protein comprising the three component product of the invention for use in the treatment of the human or animal body.

Such treatment would achieve its therapeutic effect by introduction of a nucleic acid sequence encoding an antigen for the purposes of raising an immune response. Delivery of nucleic acids can be achieved using a plasmid vector (in “naked” or formulated form) or a recombinant expression vector. To illustrate how the invention may be performed with plasmid vectors, the publication of Green T. D, et al., 2001, in Vaccine 20, 242-248 serves as an example. These authors showed that using a DNA vaccine expressing a fusion of the measles hemagglutinin protein and three copies of C3d, enhanced titers of neutralizing antibody were obtained. In the present invention, the second and third copies of C3d would be replaced with the sequence encoding the C4bp alpha chain core, resulting in an oligomeric antigen-adjuvant fusion protein. This plasmid would be smaller in size (because the core coding sequence is much shorter than that encoding two copies of C3d) and more stable because of the absence of repeated sequences.

Various viral vectors which can be utilized for gene delivery include adenovirus, herpes virus, vaccinia or an RNA virus such as a retrovirus. The retroviral vector may be a derivative of a murine or avian retrovirus. Examples of retroviral vectors in which a single foreign gene can be inserted include, but are not limited to: Moloney murine leukaemia virus (MoMuLV), Harvey murine sarcoma virus (HaMuSV), murine mammary tumour virus (MuMTV), and Rous Sarcoma Virus (RSV). When the subject is a human, a vector such as the gibbon ape leukaemia virus (GaLV) can be utilized.

The vector will include a transcriptional regulatory sequence, particularly a promoter region sufficient to direct the initiation of RNA synthesis. Suitable eukaryotic promoters include the promoter of the mouse metallothionein I gene (Hamer et al., 1982, J. Molec. Appl. Genet. 1: 273 ); the TK promoter of Herpes virus (McKnight, 1982, Cell 31: 355 ); the SV40 early promoter (Benoist et al., 1981, Nature 290: 304 ); the Rous sarcoma virus promoter (Gorman et al., 1982, Proc. Natl. Acad. Sci. USA 79: 6777); and the cytomegalovirus promoter (Foecking et al., 1980, Gene 45: 101 ).

Administration of vectors of this aspect of the invention to a subject, either as a plasmid vector or as part of a viral vector can be affected by many different routes. Plasmid DNA can be “naked” or formulated with cationic and neutral lipids (liposomes) or microencapsulated for either direct or indirect delivery. The DNA sequences can also be contained within a viral (e.g., adenoviral, retroviral, herpesvius, pox virus) vector, which can be used for either direct or indirect delivery. Delivery routes include but are not limited to intramuscular, intradermal (Sato, Y. et al., 1996, Science 273: 352-354), intravenous, intra-arterial, intrathecal, intrahepatic, inhalation, intravaginal instillation (Bagarazzi et al., 1997, J Med. Primatol. 26:27), intrarectal, intratumour or intraperitoneal.

Thus the invention includes a vector as described herein as a pharmaceutical composition useful for allowing transfection of some cells with the DNA vector such that a therapeutic polypeptide will be expressed and have a therapeutic effect, namely to induce an immune response to an antigen. The pharmaceutical compositions according to the invention are prepared by bringing the construct according to the present invention into a form suitable for administration to a subject using solvents, carriers, delivery systems, excipients, and additives or auxiliaries. Frequently used solvents include sterile water and saline (buffered or not). One carrier includes gold particles, which are delivered biolistically (i.e., under gas pressure). Other frequently used carriers or delivery systems include cationic liposomes, cochleates and microcapsules, which may be given as a liquid solution, enclosed within a delivery capsule or incorporated into food.

An alternative formulation for the administration of gene delivery vectors involves liposomes. Liposome encapsulation provides an alternative formulation for the administration of polynucleotides and expression vectors. Liposomes are microscopic vesicles that consist of one or more lipid bilayers surrounding aqueous compartments. See, generally, Bakker-Woudenberg et al, 1993, Eur. J. Clin. Microbiol. Infect. Dis. 12 (Suppl. 1): S61, and Kim, 1993, Drugs 46: 618. Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μM to greater than 10 μM. See, for example, Machy et al., 1987, LIPOSOMES IN CELL BIOLOGY AND PHARMACOLOGY (John Libbey), and Ostro et al., 1989, American J. Hosp. Phann. 46: 1576.

Expression vectors can be encapsulated within liposomes using standard techniques. A variety of different liposome compositions and methods for synthesis are known to those of skill in the art. See, for example, U.S. Pat. No. 4,844,904, U.S. Pat. No. 5,000,959, U.S. Pat. No. 4,863,740, U.S. Pat. No. 5,589,466, U.S. Pat. No. 5,580,859, and U.S. Pat. No. 4,975,282, all of which are hereby incorporated by reference.

In general, the dosage of administered liposome-encapsulated vectors will vary depending upon such factors as the patient's age, weight, height, sex, general medical condition and previous medical history. Dose ranges for particular formulations can be determined by using a suitable animal model.

Cell Culturing.

Plasmids encoding fusion proteins in accordance with the invention may be introduced into the host cells using conventional transformation techniques, and the cells cultured under conditions to facilitate the production of the fusion protein. Where an inducible promoter is used, the cells may initially be cultured in the absence of the inducer, which may then be added once the cells are growing at a higher density in order to maximise recovery of protein.

Cell culture conditions are widely known in the art and may be used in accordance with procedures known as such.

In a particular aspect, when the first component is a C4bp core protein, the fusion of at least the first two components, and where applicable, all three components, may be expressed in a prokaryotic expression system. To date, fusion proteins based on C4bp core protein have been expressed in eukaryotic cells. The yields of fusion protein from eukaryotic cells has rarely reached 2 micrograms per millilitre of culture supernatant (Oudin et al, ibid) and this could be achieved only after rounds of gene amplification. This level is too low for the economic production of large quantities of many fusion protein for therapeutic use.

Although WO91/00567 suggests that prokaryotic host cells may be used in the production of C4bp-based proteins, there is no experimental demonstration of any such production. A number of considerations however, would suggest that the use of prokaryotic systems would be disadvantageous. In particular, many eukaryotic proteins lose some or all of their active folded structure when expressed in cells such as E. coli. Other eukaryotic proteins denature or are completely inactive when expressed in prokaryotic cells.

C4bp is a secreted protein in mammals, and these are known in the art to be particularly difficult to produce in a correctly folded form in prokaryotes. Proteins with disulphide bridges are particularly problematic, as are those that require oligomerisation. Disulphide bonds are not normally produced in the reducing environment of the bacterial cytoplasm, and when they can form, they can stabilise misfolded or aggregated forms of the protein.

Usually, recombinant proteins expressed in prokaryotes are aggregated inside inclusion bodies within the host prokaryotic cell. These are discrete particles or globules separate from the rest of the cell which contain the expressed proteins usually in an agglomerated or inactive form. The presence of the expressed protein in the inclusion bodies makes it difficult to recover the protein in active soluble form as the necessary refolding techniques are techniques are inefficient and costly. Proteins purified from inclusion bodies have to be laboriously manipulated, denatured and refolded to obtain active functional proteins at relatively poor yields.

With regard to expressing C4bp core fusion proteins in prokaryotic cells, other considerations have also to be taken into account. Firstly, each core monomer retains two cysteine residues, and according to the model of C4bp multimers accepted in the art, these cysteines are required to form inter-molecular disulphide bonds during the assembly of multimers. The reducing environment of the prokaryotic cytosol such as the bacterial cytosol would be expected to prevent the formation of C4bp core multimers by reducing these disulphide bonds.

Secondly, multimers are assembled during passage through the eukaryotic secretion apparatus, which is known to assist protein folding in ways not available in prokaryotes (e.g. the presence of protein disulphide isomerase and unique chaperones). Thirdly, even under conditions where relatively small yields were obtained in eukaryotic cells (micrograms per millilitre), this secretory pathway is unable to produce homogenous protein.

Further, the inventors have also found that proteins fused to the C4bp core produced in the prokaryotic expression systems retain their functional activity. The present invention therefore provides a method for obtaining a recombinant fusion protein comprising a scaffold of a C-terminal core protein of C4bp alpha chain and a second component, and optionally a third component, said recombinant fusion protein being capable of forming multimers in soluble form in the cytosol of a prokaryotic host cell, the method including the steps of
(i) providing a prokaryotic host cell carrying a nucleic acid encoding said recombinant protein operably linked to a promoter functional in said prokaryotic cell;
(ii) culturing the host cell under conditions wherein said recombinant protein is expressed; and
(iii) recovering the recombinant protein wherein said protein is recovered in multimeric form.

We have found that the yield of protein in cell cultures of the invention can be relatively high, for example greater than 2 mg/l of culture, such as greater than 5 mg/l of culture, preferably greater than 10 mg/l of culture, such as greater than 20 mg/l culture, and even more preferably greater than 100 mg/l culture.

C4bp core fusion proteins of the invention comprise a C4bp core protein sequence fused, at the N- or C-terminus, to one of the other components of the invention. In a preferred arrangement, the order of components from the N- to C-terminal of a fusion protein is N-third component—second component—first component-C.

We have found that proteins falling within the above definition can be expressed in and recovered from bacterial expression systems in multimeric form without the need for scaffold refolding. We have expressed proteins which include C4bp core and which are capable of carrying an antigen and a second component which have a monomer weight up to about 30 kDa. The invention may thus be used to express proteins in this size range, and more generally for proteins up to about 100 kDa, more preferably about 50 kDa.

The fact that this system allows production of soluble protein in E. coli enables using it to produce, as folded soluble proteins, domains or fragments of proteins that would not fold when expressed on their own due to a lack of constraint on their C-terminal and/or N-terminal ends. Engineering a specific cleavage site enables production of the free domain of interest. Similarly constraining the N-terminal and/or C-terminal end of a peptide of interest could be beneficial during refolding processes. Furthermore, as the oligo-merisation structure is very resistant to denaturation and to disassembly, it would be stable during denaturation of the inserted protein. Therefore, during refolding, for an equal amount of protein of interest, the actual concentration of free protein would be diminished by a factor equal to the oligomerisation number. Oligomerisation may also be beneficial for purification purposes as many methods in protein technology are not optimised to work with proteins and specifically peptides of very low molecular weight.

Recovery of Protein from Culture.

Once the cells have been grown to allow for production of the protein, the protein may be recovered from the cells. Because we have found that surprisingly, the protein remains soluble, the cells will usually be spun down and lysed by sonication, for example, which keeps the protein fraction soluble and allows this fraction to remain in the supernatant following a further higher speed (e.g. 15,000 rpm for 1 hour) centrifugation.

The fusion protein in the supernatant protein fraction may be purified further by any suitable combination of standard protein chromatography techniques. We have used ion-exchange chromatography followed by gel filtration chromatography. Other chromatographic techniques, such as affinity chromatography, may also be used.

In one embodiment, we have found that heating the supernatant sample either after centrifugation of the lysate, or after any of the other purification steps will assist recovery of the protein. The sample may be heated to about 70-80° C. for a period of about 10 to 30 minutes, though this embodiment is not preferred when the second component is C3d.

Depending on the intended uses of the protein, the protein may be subjected to further purification steps, for example dialysis, or to concentration steps, for example freeze drying.

The invention is illustrated by the following examples.

### EXAMPLE 1

### Epitope-C3d-C4bp Fusion Protein

This example illustrates the fusion of an epitope (comprising amino acids 8-22 of human Cpn10) to human C3d which is itself fused to the N-terminus of the human C4bp core protein. The fusion protein was expressed in, and purified from, the bacterial strain C41(DE3). The protein behaved as an oligomer on gel filtration.

The methodology illustrated in this example may be extended to provide a three component product of the invention, for example by replacing the Cpn10 epitope with other antigen-encoding DNA in the construct described below. Alternatively, the recovered protein may be covalently linked to an antigen provided by other means.

Cloning.

A XbaI-BamHI fragment of 975 bp, (encoding the T7 ribosome binding site, residues 8-22 of human Cpn10 (the epitope) and residues 995 to 1287 of human C3d) from pAVD 95 (the expression construct for C3d7(1)in Example 2 below) was ligated into pAVD 77 (pRSETa-Db-C4bp) previously digested with XbaI and BamHI. This fused the human Cpn10 and C3d protein fragments to the C-terminal 57 residues of the alpha chain of human C4bp. The construction, called pAVD94, was checked by PCR and double digestion.

The amino acid sequence of the fusion protein of the construct is as follows:

(SEQ ID NO: 17)

MKFLPLFDRV LVERSAGSVD AERLKHLIVT PSGSGEQNMI
GMTPTVIAVH YLDETEQWEK FGLEKRQGAL ELIKKGYTQQ
LAFRQPSSAF AAFVKRAPST WLTAYVVKVF SLAVNLIAID
SQVLCGAVKW LILEKQKPDG VFQEDAPVIH QEMIGGLRNN
NEKDMALTAF VLISLQEARD ICEEQVNSLP GSITKAGDFL
EANYMNLQRS YTVAIAGYAL AQMGRLKGPL LNKFLTTAKD
KNRWEDPGKQ LYNVEATSYA LLALLQLKDF DFVPPVVRWL
NEQRYYGGGY GSTQATFMVF QALAQYQKDA PGSETPEGCE
QVLTGKRLMQ CLPNPEDVKM ALEVYKLSLE IEQLELQRDS
ARQSTLDKEL.

The residues 2-16 of SEQ ID NO:17 correspond to residues 8-22 of human Cpn10 (the epitope), residues 19-311 of SEQ ID NO:17 to human C3d residues 995 to 1287, and residues 314-370 of SEQ ID NO:17 to the 57 residues of the human C4bp core protein. A GS linker sequence, in bold in the sequence above, appears between the three components.

The protein has an estimated molecular weight of 41,485 Daltons, a theoretical pI of 5.51 and an estimated extinction coefficient of 45090 M⁻¹cm⁻¹. On this basis, to calculate the concentration we use: Abs 0.1%(=1 g/l)=1.087.

Expression.

The plasmid pAVD94 encoding the epitope-C3d-C4bp core protein was expressed in the E. coli strain C41(DE3). After overnight growth at 25° C. without induction, the protein was well expressed. After cell lysis in 20 mM Tris-HCl buffer pH8/100 mM NaCl using a French press, almost half of the protein was found to be in the supernatant.

Purification of C3d-C4bp

The soluble fraction of epitope-C3d-C4bp was purified from 1 litre of culture using three purification steps : an anion exchange column, a cation exchange column and a gel filtration column.

Anionic Column (Mono Q HR 16/10)

The column was equilibrated in 20 mM Tris-HCl buffer pH 8/100 mM NaCl. The protein was eluted with a gradient of 20 column volumes from 20 mM Tris-HCl buffer pH 8/100 mM NaCl (Buffer A) to 20 mM Tris-HCl buffer pH 8/1M NaCl (Buffer B). The protein eluted at approximately 350 mM NaCl.

The MonoQ fractions containing epitope-C3d-C4bp were dialysed against 20 mM Tris-HCl buffer pH 7/100 mM NaCl before loading on a cationic column.

Cationic Column (Mono S HR 10/10)

The fractions after the column Mono Q containing epitope-C3d-C4bp were loaded on a cationic column (Mono S HR 10/10) equilibrated with 20 mM Tris-HCl buffer pH 7/100 mM NaCl. The protein was eluted with a gradient of 20 column volumes from 20 mM Tris-HCl buffer pH 7/100 mM NaCl (Buffer A) to 20 mM Tris-HCl buffer pH 7/1M NaCl (Buffer B). The protein eluted at approximately 350 mM NaCl.

The fractions containing the epitope-C3d-C4bp without the major contaminant (>66 Kda) were pooled, concentrated and loaded on a gel filtration column.

Gel Filtration Column (Superdex 200 26/60 Prep Grade)

The fractions from the Mono S column containing epitope-C3d-C4bp were loaded on a Gel Filtration column (Superdex 200 26/60 prep grade) equilibrated with 50 mM Na phosphate pH 7.4/150 mM NaCl. The protein eluted with 152.69 ml of buffer as a nice symmetric peak. This elution volume shows that the protein is oligomeric. After the column, the protein concentration was 0.45 mg/ml. The protein was concentrated to 1.5 mg/ml and stored at −70° C. with 10% Glycerol. The protein was at least 90% pure.

### EXAMPLE 2

### Insertion of the Human C3d Molecule in the Mobile Loop of Human Cpn10 (C3d7)

This example describes the purification of the soluble portion of three similar C3d7 constructs and their expression at 25° C.

C3d7(1)

A 42.85 kDa tri-partite fusion protein, comprising human C3d replacing the mobile loop of human Cpn10 (truncated at its N-terminus) and a C-terminal myc tag epitope, with the amino acid sequence. SEQ ID NO:18, was expressed from the plasmid pAVD 95 in the E. coli strain C41(DE3) at 25° C.

(SEQ ID NO: 18)

MKFLPLFDRV LVERSAGSVD AERLKHLIVT PSGSGEQNMI
GMTPTVIAVH YLDETEQWEK FGLEKRQGAL ELIKKGYTQQ
LAFRQPSSAF AAFVKRAPST WLTAYVVKVF SLAVNLIAID
SQVLCGAVKW LILEKQKPDG VFQEDAPVIH QEMIGGLRNN
NEKDMALTAF VLISLQEAKD ICEEQVNSLP GSITKAGDFL
EANYMNLQRS YTVAIAGYAL AQMGRLKGPL LNKFLTTAKD
KNRWEDPGKQ LYNVEATSYA LLALLQLKDF DFVPPVVRWL
NEQRYYGGGY GSTQATFMVF QALAQYQKDA PGSGKVLQAT
VVAVGSGSKG KGGEIQPVSV KVGDKVLLPE YGGTKVVLDD
KDYFLFRDGD ILGKYVDeqk liseedl

Human Cpn10 amino acid sequence of SEQ ID NO:18 are residues 1-16 and 311-377. The human C3d amino acid sequence is from 17-310 of SEQ ID NO:18, and the myc-tag epitope amino acid sequence from 378-387.

The DNA sequence (an NdeI-HindIII restriction fragment) encoding this fusion protein was cloned between the NdeI-HindIII sites of a pRSET derived plasmid, placing the coding sequence under the control of the T7 promoter.

C3d7 (2)

A second fusion protein, differing only in the positioning of human C3d insertion in place of the mobile loop of human Cpn10 was similarly constructed. This has the sequence of SEQ ID NO:19:

(SEQ ID NO: 19)

MKFLPLFDRV LVERSAGETV TVDAERLKHL IVTPSGSGEQ
NMIGMTPTVI AVHYLDETEQ WEKFGLEKRQ GALELIKKGY
TQQLAFRQPS SAFAAFVKRA PSTWLTAYVV KVFSLAVNLI
AIDSQVLCGA VKWLILEKQK PDGVFQEDAP VIHQEMIGGL
RNNNEKDMAL TAFVLISLQE AKDICEEQVN SLPGSITKAG
DFLEANYMNL QRSYTVAIAG YALAQMGRLK GPLLNKFLTT
AKDKNRWEDP GKQLYNVEAT SYALLALLQL KDFDFVPPVV
RWLNEQRYYG GGYGSTQATF MVFQALAQYQ KDAPGKVLQA
TVVAVGSGSK GKGGEIQPVS VKVGDKVLLP EYGGTKVVLD
DKDYFLFRDG DILGKYVDeq kliseedl

Amino acid residues 1-20 and 315-378 are derived from human Cpn10, flanking the human C3d amino acid sequence. The myc-tag epitope amino acid sequence is from 379-388.

C3d7 (3)

Likewise, a third fusion protein called C3d7(3) with the following amino acid sequence was also produced:

(SEQ ID NO: 20)

MKFLPLFDRV LVERSAGETV DAERLKHLIV TPSGSGEQNM
IGMTPTVIAV HYLDETEQWE KFGLEKRQGA LELIKKGYTQ
QLAFRQPSSA FAAFVKRAPS TWLTAYVVKV FSLAVNLIAI
DSQVLCGAVK WLILEKQKPD GVFQEDAPVI HQEMIGGLRN
NNEKDMALTA FVLISLQEAK DICEEQVNSL PGSITKAGDF
LEANYMNLQR SYTVAIAGYA LAQMGRLKGP LLNKFLTTAK
DKNRWEDPGK QLYNVEATSY ALLALLQLKD FDFVPPVVRW
LNEQRYYGGG YGSTQATFMV FQALAQYQKD APLQATVVAV
GSGSKGKGGE IQPVSVKVGD KVLLPEYGGT KVVLDDKDYF
LFRDGDILGK YVDeqklise edl

The human Cpn10 amino acid sequence is 1-18 and 313-373 flanking the human C3d amino acid sequence, and the myc-tag epitope amino acid sequence is 374-383.

Expression of C3d7 (1).

To have this protein in a soluble form, we expressed pAVD95 in the E. coli strain C41(DE3) at 25° C. After an overnight induction at 25° C. with 0.5 mM IPTG the protein was expressed and almost half of the protein was found to be in the supernatant.

Purification

The soluble fraction of C3d7(1) was purified using two purification steps, namely an anionic column, followed by a gel filtration column.

Anionic Column (Mono Q HR 16/10)

The column was equilibrated in 20 mM Tris pH 8. The protein was eluted with a gradient of 20 column volumes from 20 mM Tris pH 8 to 20 mM Tris pH 8, 1M NaCl. The protein eluted with approximately 350 mM NaCl in one fraction (E3) of 5 ml.

Gel Filtration Column (Superdex 200 26/60 Prep Grade)

The fraction E3 of the column Mono Q was loaded on a gel Filtration column (Superdex 200 26/60 prep grade) equilibrated with 50 mM Na phosphate pH 7.4, 150 mM NaCl. The protein was eluted with 150 ml of buffer. The elution volume of ovalbumin (MW=43 Kd) on the same column was 167 ml. This indicates that the protein is oligomeric.

Circular Dichroism

Analysis of the protein by Far UV Circular Dichroism indicated the presence of secondary structure. The deconvolution of the spectrum gave a percentage of alpha-helix around 49%. This percentage is in agreement with the percentage determined by modelling (48% of alpha-helix). This-is a good indication that the protein is correctly folded.

The protein was concentrated to 1.2 mg/ml in 50 mM sodium phosphate, pH7.4, 150 mM NaCl.

### EXAMPLE 3

### CR2 Binding Activity of C3d7(1) and Epitope-C3d-C4bp

ELISA Assay Method

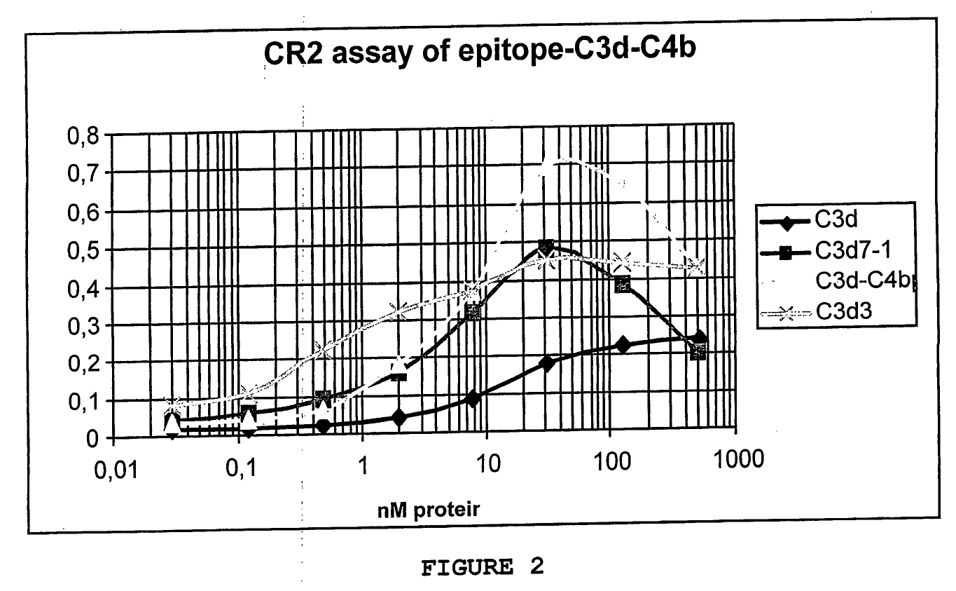

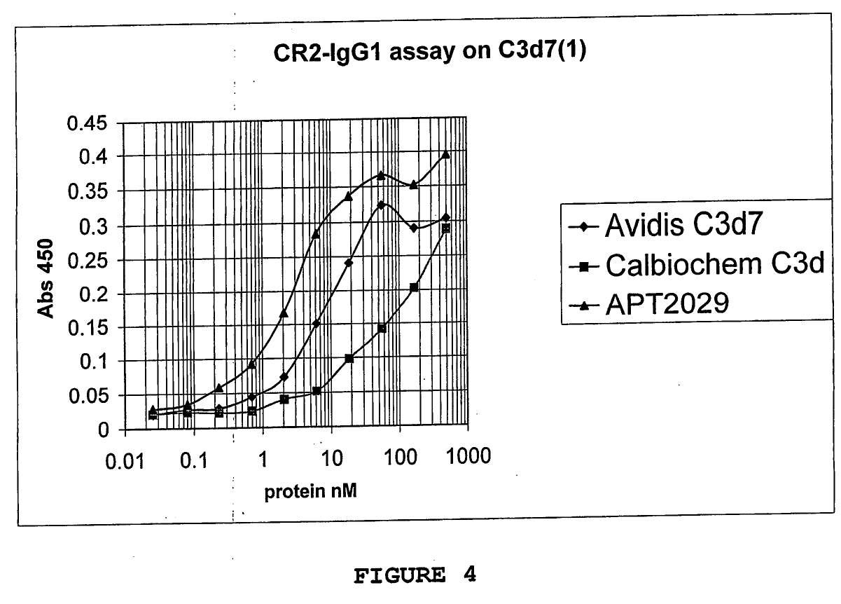

The epiotope-C3d-C4bp molecule prepared as in Example 1 and the C3d7(1) prepared as in Example 2 were assayed over a concentration range from 500 nM-0.01 nM and compared against human C3d (Calbiochem) and a linear trimer of human C3d, called C3d3 or APT2029, constructed and prepared as described in WO99/35260. The results are shown in FIGS. 2 and 4.

Briefly, the assay method was as follows:

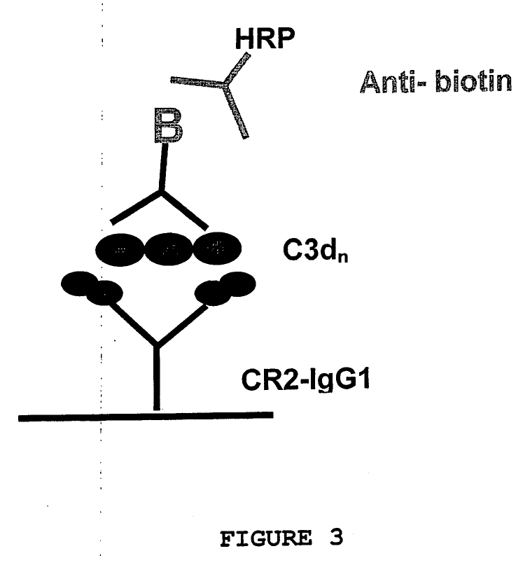

A IgG constant region-CD21 fusion protein was expressed and purified in tissue culture cells and the purified protein was used to coat the wells of an ELISA plate. The various C3d molecules were added, in a range of concentrations, to these wells and incubated. After incubation, the wells were extensively washed, before adding a biotinylated anti-C3d monoclonal antibody. After incubation and washing, a horseradish peroxidase(HRP)-labelled anti-biotin antibody was added. Following a further incubation and washing step, a substrate for HRP was added and the generation of a coloured product from the substrate by the HRP was measured at an absorbance of 450 nanometres. The assay is illustrated as a cartoon in FIG. 3.

Clearly the epitope-C3d-C4bp molecule binds to the C3d receptor CD21 much better than the monomeric C3d does and better than even the linear trimer C3d3 at several concentrations, as seen in FIG. 2.

The assay was repeated three times with C3d7(1) and the gradients of each response averaged and compared. FIG. 4 shows the results of one of these assays.

In comparing the results between C3d7(1) and the Calbiochem C3d (which is in monomeric form), the Abs 450 of the C3d7(1) increases at a lower concentration than the monomeric C3d. This indicates that the C3d is in a multimeric form.

### EXAMPLE 4

### CR2 Binding Activity of C3d7(1), (2) & (3)

The second binding experiment of example 3 was repeated as described in Example 3 with C3d7(1), (2) and (3), all of which were prepared as described above in Example 2.

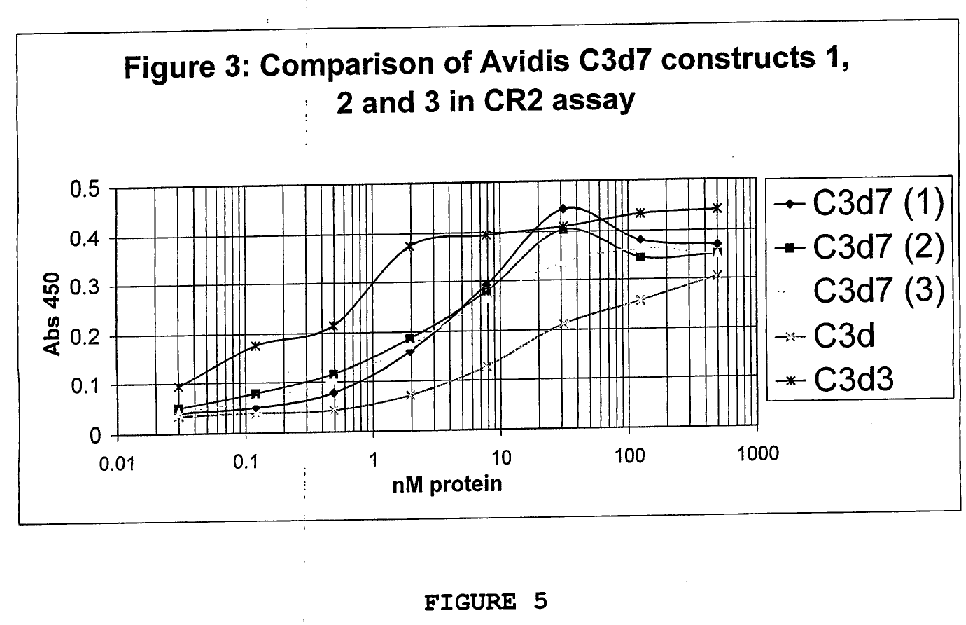

The binding of these three proteins in an ELISA assay is shown as FIG. 5. The data show that C3d7(1), C3d7(2) and C3d7(3) all conclusively bind to CR2. The gradient of the linear portion of the binding curve gives an indication of multimerisation of the proteins as shown by the 3.4 fold increase in gradient between the monomeric C3d (supplied by Calbiochem) and the linear trimer of C3d3, called APT2029. The gradient for the linear portion of the three C3d7 constructs suggests that they are all multimerised.

### EXAMPLE 5

### Analysis by Immunofluorescent Flow Cytometry.

The CR2 binding activity of the C3d7 constructs on the immortalised human CD21+ Raji lymphoblastoid cell line and human CD20⁺/CD4⁻/CD8⁻ peripheral blood lymphocytes was tested. Flow cytometry analysis was carried out using a Becton Dickenson FACSCalibur; 10,000 events were acquired.

Immortalised Raji (B cells) and Jurkat (T cells) cells were washed in PBS and incubated with optimised dilutions of FITC conjugated anti-human, CD3 (pan-T cell marker), CD20 (pan-B cell marker) and CD21 (CR2 marker) (DAKO) monoclonal antibodies (Mabs). This verified that Raji cells are CD21⁺, CD20⁺ whereas Jurkat cells are CD21^{dim}, CD3⁺.

Binding of C3d7 (1-3) is Detected on Raji (CD21⁺/CD20⁺) Cells, but not Jurkat (CD3⁺/CD21^{dim}) Cells.

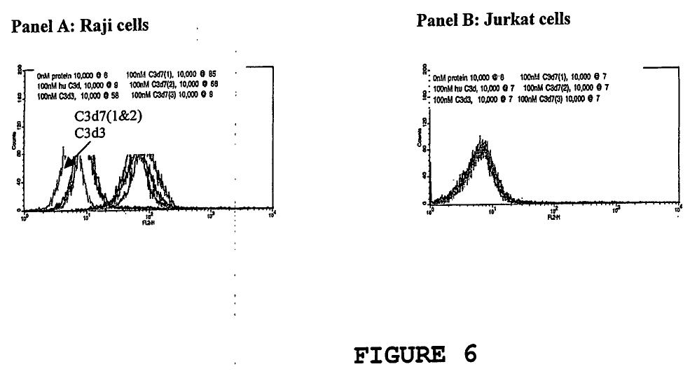

Using a single staining immunofluorescence assay format, washed Raji and Jurkat cells (1×10⁶/ml) were incubated with 100 nM (final dilution) of C3d7(1), C3d7(2), C3d7(3), human monomeric C3d (Calbiochem) and human linear trimer C3d3 (APT2029) for 30 minutes at room temperature, washed in ice-cold PBS and then incubated with an optimised dilution of Cy3 (a pink fluorophore) conjugated anti-human C3d monoclonal antibody (Mab) for 30 minutes at 4° C. in the dark, washed again and resuspended in 0.5 ml ice-cold PBS. FIG. 6 shows the results of this analysis.

C3d7(1) and C3d7(2) conclusively bind to CD21⁺ cells and not to CD3⁺/CD21^{dim} cells. The increase in signal intensity gives an indication of multimerisation shown by the 7 and 9 fold signal intensity increase between C3d (Calbiochem), C3d7(1) and C3d7(2) respectively compared with the 6.4 fold increase with C3d₃ (APT2029).

Binding of C3d7 (1) on the Surface of CD20⁺/CD4⁻/CD8⁻ Human Peripheral Blood Lymphocytes (PBLs)

This experiment was conducted using a double staining immunofluorescence assay format. Human PBLs were isolated from blood by density gradient centrifugation through Ficoll. Contaminating erythrocytes were removed by lysis. Washed PBLs at 1×10⁶/ml were incubated with 200 nM (final dilution) C3d7(1) or human linear trimer C3d3 (APT2029) for 30 minutes at room temperature, washed in ice-cold PBS and then incubated with optimised dilutions of Cy3-anti-human C3d Mab and FITC-anti CD4 (Th cell marker), anti CD8 (CTL marker) anti-CD20 (B cell marker) Mabs (DAKO) for 30 minutes at 4° C. in the dark, washed and resuspended in 0.3 ml ice-cold PBS prior to flow cytometry analysis; 5,000 events were acquired.

Analysis of the data indicated that C3d7(1) conclusively binds to the PBL B (CD20⁺) cell population (presumed to be CD21⁺), in a similar manner to that seen with the linear trimer human C3d3 called APT2029.

1. A product comprising:
a first component which is a scaffold;
a second component which is an adjuvant; and
a third component which is an antigen.

2. A product according to claim 1 wherein the second component is a polypeptide which is a ligand for CD21 or a cell surface molecule on B cells or T cells or follicular dendritic or other antigen presenting cells

3. A product according to claim 1 wherein the third component is a polypeptide antigen.

4. A product according to claim 1 wherein the third component is a non-polypeptide antigen.

5. A product according to claim 1 wherein the scaffold and antigen are the same.

6. A product according to claim 5 wherein the scaffold and antigen are a viral coat protein.

7. A product according to claim 6 wherein the viral coat protein is Hepatitis B surface antigen.

8. A product according to claim 1 wherein the scaffold and adjuvant are the same.

9. A product according to claim 8 wherein the scaffold and adjuvant are C4bp core protein.

10. A pharmaceutical composition comprising the product of claim 1 together with a pharmaceutically acceptable carrier or diluent.

11. A method of inducing an immune response to an antigen which method comprises administering to a subject an effective amount of a product according to claim 1.

12. A method of making a product comprising:
a first component which is a polypeptide scaffold;
a second component which is a polypeptide which is a ligand for CD21 or a cell surface molecule on B cells or T cells or follicular dendritic or other antigen presenting cells; and
a third component which is a polypeptide antigen,
the method comprising expressing nucleic acid encoding the three components in the form of a fusion protein, and recovering the product.

13. A method of making a product comprising:
a first component which is a polypeptide scaffold;
a second component which is a polypeptide which is a ligand for CD21 or a cell surface molecule on B cells or T cells or follicular dendritic or other antigen presenting cells; and
a third component which is a non-polypeptide antigen,
the method comprising expressing nucleic acid encoding the first and second components in the form of a fusion protein, joining said fusion protein to the third component, and recovering the product.

14. The method of claim 12 wherein the nucleic acid is expressed in a prokaryotic host cell.

15. A method according to claim 14 wherein the fusion protein is recovered in multimeric form.

16. A method according to claim 15 wherein the recombinant protein is present at least at a concentration of at least 2 mg/l of cell culture.

17. A method according to claim 15 wherein the host prokaryotic cell is E. coli.

18. An expression vector comprising a nucleic acid sequence encoding a fusion protein of
a first component which is a polypeptide scaffold;
a second component which is a polypeptide which is a ligand for CD21 or a cell surface molecule on B cells or T cells or follicular dendritic or other antigen presenting cells; and optionally
a third component which is a polypeptide antigen,
operably linked to a promoter functional in a host cell.

19. A bacterial host cell transformed with the expression vector of claim 18.

20. A eukaryotic host cell transformed with the vector of claim 18.

21. Use of the expression vector of claim 20 in a method of treatment of the human or animal body.


SEQUENCE LISTING (20 sequences)

SEQ ID NO: 1 - protein, 57 aa, Homo sapiens
ETPEGCEQVLTGKRLMQCLPNPEDVKMALEVYKLSLEIEQLELQRDSARQSTLDKEL

SEQ ID NO: 2 - protein, 57 aa, Oryctolagus cuniculus
EVPEGCEQVQAGRRLMQCLADPYEVKMALEVYKLSLEIELLELQRDKARKSSVLRQL

SEQ ID NO: 3 - protein, 55 aa, Rattus sp.
EVPKDCEHVFAGKKLMQCLPNSNDVKMALEVYKLTLEIKQLQLQIDKAKHVDREL

SEQ ID NO: 4 - protein, 54 aa, Mus sp.
EASEDLKPALTGNKTMQYVPNSHDVKMALEIYKLTLEVELLQLQIQKEKHTEAH

SEQ ID NO: 5 - protein, 67 aa, Bos sp.
EYPEGCEQVVTGRKLLQCLSRPEEVKLALEVYKLSLEIEILQTNKLKKEAFLLREREKNVTCDFNPE

SEQ ID NO: 6 - protein, 57 aa, Sus scrofa
EYPEDCEQVHEGKKLMECLPTLEEIKLALALYKLSLETNLLELQIDKEKKAKAKYST

SEQ ID NO: 7 - protein, 56 aa, Cavia porcellus
EVPEECKQVAAGRKLLECLPNPSDVKMALEVYKLSLEIEQLEKEKYVKIQEKFSKE

SEQ ID NO: 8 - protein, 59 aa, Mus sp.
EVLEDCRIVSRGAQLLHCLSSPEDVHRALKVYKLFLEIERLEHQKEKWIQLHRKPQSMK

SEQ ID NO: 9 - protein, 52 aa, Artificial Sequence
  Description of Artificial Sequence Variant of the C4bp core protein
CEQVLTGKRLMQCLPNPEDVKMALEVYKLSLEIEQLELQRDSARQSTLDKEL

SEQ ID NO: 10 - protein, 57 aa, Artificial Sequence
  Description of Artificial Sequence Variant of the C4bp core protein
ETPEGCEQVLTGKRLMQCLPNPEDVKMALEVYKLSLEIKQLELQRDSARQSTLDKEL

SEQ ID NO: 11 - protein, 52 aa, Artificial Sequence
  Description of Artificial Sequence Variant of the C4bp core protein
CEQVLTGKRLMQCLPNPEDVKMALEVYKLSLEIKQLELQRDSARQSTLDKEL

SEQ ID NO: 12 - protein, 57 aa, Artificial Sequence
  Description of Artificial Sequence Variant of the C4bp core protein
ETPEGCEQVLTGKRLMQCLPNPEDVKMALEIYKLSLEIEQLELQRDSARQSTLDKEL

SEQ ID NO: 13 - protein, 57 aa, Artificial Sequence
  Description of Artificial Sequence Variant of the C4bp core protein
ETPEGCEQVLTGKRLMQCLPNPEDVKMALEIYKLSLEIKQLELQRDSARQSTLDKEL

SEQ ID NO: 14 - protein, 50 aa, Artificial Sequence
  Description of Artificial Sequence Variant of the C4bp core protein
EGCEQALTGKRLMQCLPNPEDVKMALEIYKLSLEIKQLELQRDSARQSTL

SEQ ID NO: 15 - protein, 57 aa, Artificial Sequence
  Description of Artificial Sequence Variant of the C4bp core protein
ETPEGSEQVLTGKRLMQSLPNPEDVKMALEVYKLSLEIKQLELQRDSARQSTLDKEL

SEQ ID NO: 16 - protein, 52 aa, Artificial Sequence
  Description of Artificial Sequence Variant of the C4bp core protein
EGSEQALTGKRLMQSLPNPEDVKMALEIYKLSLEIEQLELQRDSARQSTLDK

SEQ ID NO: 17 - protein, 370 aa, Artificial Sequence
  Description of Artificial Sequence Fusion Protein
MKFLPLFDRVLVERSAGSVDAERLKHLIVTPSGSGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEARDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPGSETPEGCEQVLTGKRLMQCLPNPEDVKMALEVYKLSLEIEQLELQRDSARQSTLDKEL

SEQ ID NO: 18 - protein, 387 aa, Artificial Sequence
  Description of Artificial Sequence Fusion Protein
MKFLPLFDRVLVERSAGSVDAERLKHLIVTPSGSGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPGSGKVLQATVVAVGSGSKGKGGEIQPVSVKVGDKVLLPEYGGTKVVLDDKDYFLFRDGDILGKYVDEQKLISEEDL

SEQ ID NO: 19 - protein, 388 aa, Artificial Sequence [sequence omitted]

SEQ ID NO: 20 - protein, 383 aa, Artificial Sequence
  Description of Artificial Sequence Fusion Protein
MKFLPLFDRVLVERSAGETVDAERLKHLIVTPSGSGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPLQATVVAVGSGSKGKGGEIQPVSVKVGDKVLLPEYGGTKVVLDDKDYFLFRDGDILGKYVDEQKLISEEDL